(19) United States
(12) Patent Application Publication
(10) Pub. No.: US 2026/0018281 A1
(43) Pub. Date: Jan. 15, 2026

(54) ANOMALY DETECTION AND ADAPTIVE NOTIFICATION SYSTEM

(71) Applicant: Panasonic Well LLC, Palo Alto, CA (US)

(72) Inventor: Ramin Firoozye, San Francisco, CA (US)

(21) Appl. No.: 19/240,628

(22) Filed: Jun. 17, 2025

Related U.S. Application Data
(60) Provisional application No. 63/670,632, filed on Jul. 12, 2024.

(51) Int. Cl. G16H 40/63

(52) U.S. Cl. CPC G16H 40/63

(57) ABSTRACT

Health monitoring methods and systems are described. Methods include receiving values for characteristics of an individual. The characteristics may include physical properties or medical history properties of the individual. Methods may further include selecting a profile for the individual. Selecting includes using the values of the characteristics. The profile may specify expected ranges for event data. Methods may also include configuring a health monitoring application with the profile to generate notifications following an event exceeding an expected range. Methods may include collecting an event data set and a notification data set from the individual. In addition, methods may include customizing the profile for the individual to form a custom profile. Customizing may include using a trained machine learning model to process a data set to output accurate notifications. Methods may include configuring the health monitoring application with the custom profile.

### CROSS-REFERENCES TO RELATED APPLICATIONS

The present patent application claims priority to U.S. Provisional Patent Application No. 63/670,632 filed Jul. 12, 2024, the disclosures of which are incorporated herein by reference in its entirety for all purposes.

### BACKGROUND

Smartphone health monitoring applications and other health monitoring applications have increased in popularity as a result of the widespread use of mobile technology and growing interest in personal wellness. These apps utilize the sensors found in modern smartphones—such as accelerometers, gyroscopes, GPS, cameras, and biometric sensors—to track various aspects of a user's health and activity. Functionalities include tracking physical activity, monitoring heart rate, analyzing sleep patterns, and providing reminders for tasks.

Despite their potential benefits, current smartphone health monitoring apps exhibit significant shortcomings. The accuracy and reliability of these apps can be inconsistent, as they often rely on smartphone sensors that vary in quality. Data integration and interoperability issues further limit their effectiveness, as health data are frequently siloed and difficult to share across platforms. Additionally, user engagement tends to wane over time due to repetitive data entry, lack of personalized feedback, and perceived effort required for consistent app use. Furthermore, health monitoring applications typically interact with a single individual. The data collected may be of initial interest to the user but may need active analysis by the user or a medical practitioner to understand more about the overall health of the user.

The present disclosure addresses shortcomings of present health monitoring applications by improving detection of anomalous events and facilitating a notification workflow of users and caregivers.

### BRIEF SUMMARY

The present disclosure may monitor the health of an individual and send notifications when an anomalous event is detected. A health monitoring application may select a baseline profile for a particular individual based on the individual's information and sensor data. In some embodiments, the selected baseline profile may not be sufficiently accurate for detecting anomalous events and sending notifications. A custom profile can be generated for the individual. The custom profile may be generated through training a machine learning model using the individual's information and/or sensor data. The customized profile can be used to detect anomalous events. Upon detecting anomalous events, notifications may be sent to the individual and/or other suitable contacts. The adaptability of the notification system and the increased accuracy from a custom profile may lead to better clinical outcomes, saving lives and providing better care at a lower cost.

In embodiments, a computer-implemented method includes receiving values for characteristics of an individual. The characteristics include physical properties or medical history properties of the individual. The method may further include selecting a profile for the individual. Selecting includes using the values of the characteristics. The profile may specify expected ranges for event data. The method may also include configuring a health monitoring application with the profile to generate notifications following an event exceeding an expected range. Additionally, the method may include collecting an event data set and a notification data set from the individual. The method may include customizing the profile for the individual to form a custom profile. Customizing includes using a trained machine learning model to process a data set to output accurate notifications. The data set includes the event data set, the notification data set, and a set of labels indicating the accuracy of notifications in the notification data set. The method may include configuring the health monitoring application with the custom profile.

Embodiments include a computer-readable medium on which computer-executable instructions are stored to implement a method. The method includes receiving values for characteristics of an individual. The characteristics include physical properties or medical history properties of the individual. The method may further include selecting a profile for the individual. Selecting includes using the values of the characteristics. The profile may specify expected ranges for event data. The method may also include configuring a health monitoring application with the profile to generate notifications following an event exceeding an expected range. Additionally, the method may include collecting an event data set and a notification data set from the individual. The method may include customizing the profile for the individual to form a custom profile. Customizing includes using a trained machine learning model to process a data set to output accurate notifications. The data set includes the event data set, the notification data set, and a set of labels indicating the accuracy of notifications in the notification data set. The method may include configuring the health monitoring application with the custom profile.

Embodiments include a computing system. The computing system may include a processor and a memory storing instructions configured such that, when executed in cooperation with controlling the processor, the instructions operate the computing system to perform a method. The method may be the computer-implemented method described herein.

A better understanding of the nature and advantages of embodiments of the present invention may be gained with reference to the following detailed description and the accompanying drawings.

### Terms and Definitions

The terms “cutoff” and “threshold” refer to predetermined numbers used in an operation. For example, a threshold accuracy can refer to an accuracy of a health monitoring profile below which a profile is not applied. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. A cutoff may be chosen after and based on output of the test data. A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

“Machine learning model” refers to a software module configured to be run on one or more processors to provide a classification or numerical value of a property of one or more samples. A machine learning (ML) model can be generated using sample data (e.g., training data) to make predictions on test data. One example is an unsupervised learning model. Another example type of model is supervised learning that can be used with embodiments of the present disclosure. Example supervised learning models may include different approaches and algorithms including analytical learning, statistical models, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naïve Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, K-Nearest Neighbors (KNN), average and median KNN, Gaussian Mixture Model (GMM), isolation forest (iForest), local outlier detection (LOD), ball tree, random-cut forest (RCF), cascading K-means clustering, C.45 decision tree, clustered hierarchic anomaly and outlier detection (CHAODA), copula-based outlier detection (COPOD), streaming HS-trees, or Proaftn, a multicriteria classification algorithm. The model may include linear regression, logistic regression, deep recurrent neural network (e.g., long short-term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an overview of a process of provisioning a new user and detecting an anomaly according to embodiments of the present invention.

FIG. 2 illustrates an overview of generating a notification event according to embodiments of the present invention.

FIG. 3 illustrates an overview of a notification system 300 for a notification event 302 according to embodiments of the present invention.

FIG. 4 illustrates operations performed by various components according to embodiments of the present invention.

FIG. 5 illustrates a method 500 for monitoring the health of an individual according to embodiments of the present invention.

FIG. 6 illustrates a machine learning architecture 600 according to embodiments of the present invention.

FIG. 7 illustrates a computing system architecture 700 according to embodiments of the present invention.

### DETAILED DESCRIPTION

Ordinary methods of monitoring health include regular checkups with a medical practitioner. These checkups may be at yearly or less frequent intervals. These visits also provide a limited and non-representative view of an individual's life. Ordinary fitness trackers may monitor basic information, such as heartbeat, steps, blood oxygen level, but do not provide an understanding of the health of the individual nor inform the individual if a serious medical event is occurring or about to occur. Such fitness trackers may not have mechanism to notify relevant contacts for a health issue.

Embodiments of the present invention may include determining a profile for an individual. The profile can be used to detect anomalous events that may indicate a health issue. User input, sensor data, or ambient data can be collected to assist with detecting events associated with an individual. The input and data can create activity streams. These activity streams can be analyzed to determine patterns and to detect anomalies.

User-generated events may be based on user interactions and a series of environmental and personal sensor readings. These may be considered raw events. The raw data of the raw events may be characterized, simplified, and/or transformed so that a synthetic event is being analyzed. A synthetic event or a combination of synthetic events can be used to indicate life events. These life events may be grouped into categories. Associated with these events are metadata, such as start/end times, location, state (e.g., on/off, asleep/awake), and levels (e.g., heart rate, light brightness value), which can be used to indicate life events.

The events may offer a high-level picture of a typical person's day-to-day activities. They can be stored as both current state and a changelog associated with a Human Digital Twin. The series of events can be used to train a model. The data can be used to determine a baseline. Subsequent events can be gauged against this baseline for the degree of variance from the norm. An event anomaly detection engine can then provide an indicator of degree of variance and a heuristic system can decide whether that value is sufficiently abnormal to merit notification.

The data stream can also be used to adjust the training model to allow for variations in one's routines. A training model may be generalized and used for other users who fall within a similar demographic as a baseline. The demographic may include age, gender, location, ethnicity, nationality, past health history, past activity history, or other characteristics. For example, a demographic may be a 75-year-old woman, and a baseline may include the typical sleep patterns of the 75-year-old woman.

This base model may establish a common baseline but an individual's habits may still deviate from that baseline without being anomalous events. In that case, the model can be discarded or adjusted, and a custom training model can be created to match the individual. The custom training model may be created using machine learning and/or human intervention.

Anomalous events can be detected using a custom baseline from the custom training model. Upon detection of the anomalous event, a health monitoring system may notify appropriate contacts. These contacts may be the individuals themselves, friends, family, caregivers, medical professionals, and/or emergency responders. The notification may vary depending on the severity of the detected event.

Embodiments of the present invention may improve the health outcomes of individuals, including saving lives. Embodiments also provide a better understanding of the health picture of individuals using data and a baseline for the individual.

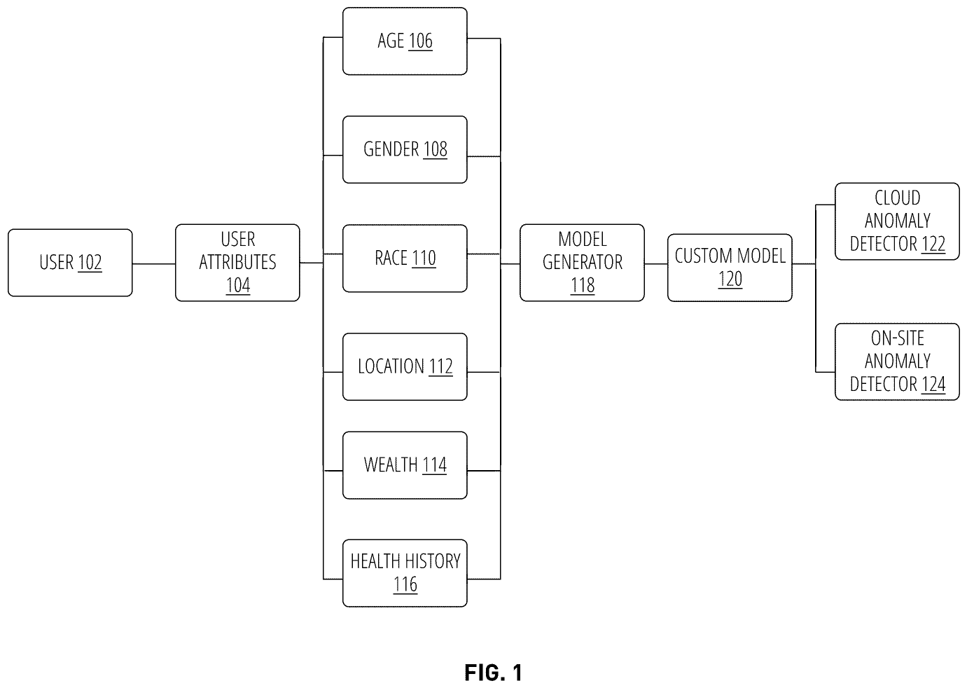

FIG. 1 shows an overview of a process of provisioning a new user 102 and detecting an anomaly. User 102 may be provisioned based on user attributes 104. User attributes 104 may include, but are not limited to, age 106, gender 108, race 110, location 112, wealth 114, and/or health history 116. Other user attributes may include socio-economic status, activity level, proximity to friends/family, mental health state, current slate of medications, occupation, education level, marital status, housing and living conditions, access to healthcare, lifestyle factors (e.g., diet, physical activity, smoking, alcohol consumption, drug use), or family health history. User attributes may be physical properties or medical history properties.

The user attributes 104 may be inputted into model generator 118. The model generator 118 can determine a base model for user 102, using the provisioning. The base model may be based on reference individual(s) who have the same or similar attributes as user attributes 104. For example, values or categories for user attributes 104 may be within a certain percentage (e.g., 5%, 10%, 20%, 30%, 40%, or 50%) of the values or categories for the reference individual(s). As another example, a certain amount (number or percentage) of user attributes 104 may be within a certain percentage of the reference individual(s). All or most (e.g., at least 50%, 60%, 70%, 80%, or 90%) of the attribute categories may be similar to the reference individual(s). Some user attributes may be weighted higher than other user attributes such that similarity with certain attributes is more important for selecting the base model than other attributes.

In embodiments, the base model may result from an actual reference individual or averages of actual reference individuals with the same or similar attributes. In other embodiments, the base model may be based on a combination of different reference individuals with different attributes. For example, a user may be provisioned with attributes of a Japanese woman, over the age of 70, history of high blood pressure, and active in doing Pilates twice a week. If no reference individual exists with all these categories, a base model may be generated from one reference individual having some of the attributes and another reference individual having other attributes. In some embodiments, the base model may involve extrapolating or interpolating the effects of attributes. Weighted averages of different individuals may be used depending on a similarity score with user 102. Generating the model may involving approximating the effect of one attribute on an individual. For example, generating the model may be able to approximate the effect of doing Pilates twice a week on a Japanese woman, over the age of 70, with a history of high blood pressure.

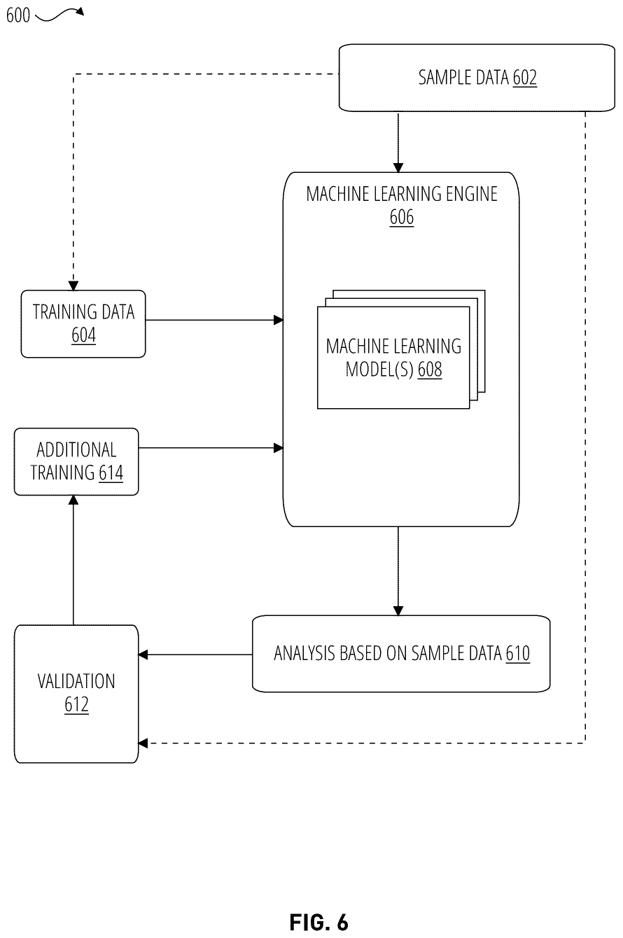

Generating the base model may include using machine learning, as described with machine learning architecture 600 in FIG. 6. A machine learning model may be trained using data sets including profiles from reference profiles and the eventual base model or custom model used for different test individuals. The machine learning model may output a base model when user attributes 104 are input into the machine learning model.

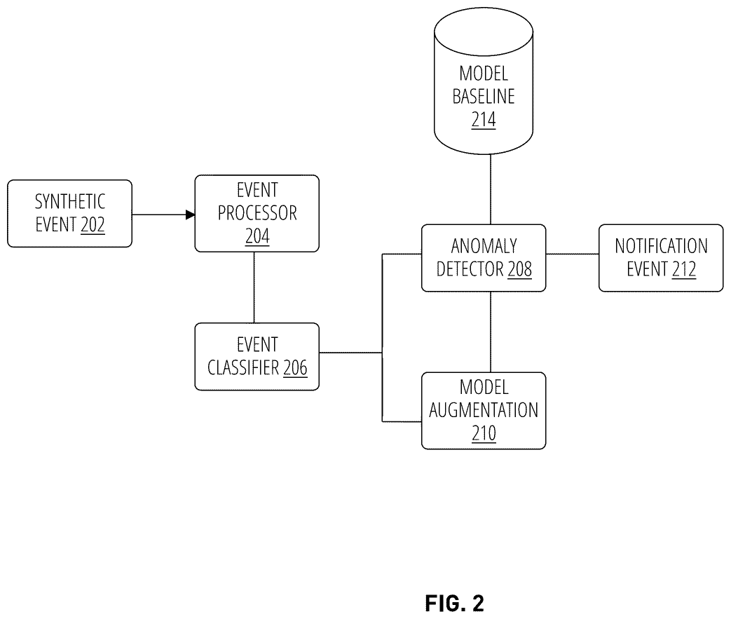

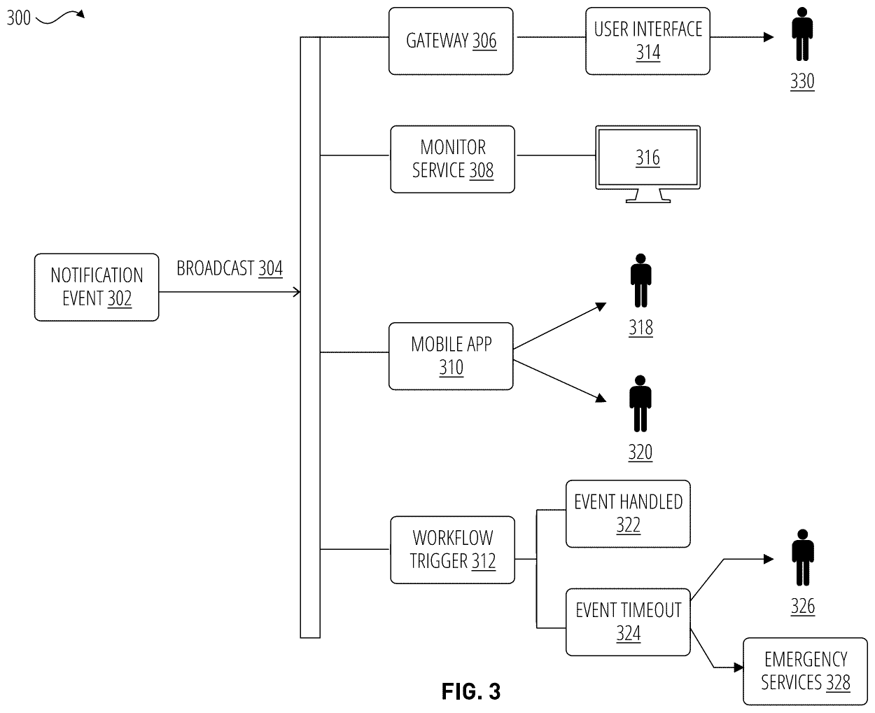

In some embodiments, model generator 118 generates custom model 120. A system may monitor the number of false-negative notifications using a user feedback loop and trigger a new training process. Notifications are described in more detail with FIG. 2 and FIG. 3. The new training process can be used to adjust for an individual who has undergone a major life change (e.g., surgery, medical, mental trauma, etc.) and re-create a baseline more appropriate to their current state. In some embodiments, the individual has not undergone a major life change event but the base model does not provide a suitable baseline for user 102. Custom model 120 may be generated with a machine learning model and is described in more detail with model augmentation 210 in FIG. 2.

Custom model 120 may be accessed by a cloud anomaly detector 122 and/or an on-site anomaly detector 124. Both these anomaly detectors may determine when an event is sufficiently different from a baseline of custom model 120. An event may be considered anomalous when input or sensor data values is significantly different from the corresponding input or sensor data of the baseline. In embodiments, an event may be considered anomalous when a certain number of inputs or sensors have data values that are different but may not be significantly different from the baseline. Anomaly detection is described in more detail with anomaly detector 208 in FIG. 2.

A cloud anomaly detector 122 may be in a different geographic location (e.g., different building, different city, state/province, country) than user 102. An on-site anomaly detector 124 may be located in the same building or room as user 102. The on-site anomaly detector 124 may be executed by software on a mobile device (e.g., cell phone) of user 102. In embodiments, on-site anomaly detector 124 may reside on a gateway, mobile phone, tablet, or other dedicated device. Cloud anomaly detector 122 and on-site anomaly detector 124 may share responsibilities for detecting an anomaly. For example, on-site anomaly detector 124 may receive sensor data while cloud anomaly detector 122 may perform analysis on the data.

FIG. 2 illustrates an overview of generating a notification event. A synthetic event 202 may occur. A synthetic event 202 may be the simplified or transformed data of a raw event, after one or more statistical or mathematical operations. For example, the raw data may be the heart rate, and synthetic event 202 may be the median of heart rate during daytime hours. As other examples, synthetic event 202 may be represented by minimum, maximum, percentile, first derivative, second derivative, shape score, mode, mean, frequency, period, wavelength, or a combination thereof. For example, synthetic event 202 may be represented by the median value over a sliding time window. Synthetic event 202 may be represented by a vector or matrix of multiple values, including for example, 10 to 100 values, 100 to 1,000 values, 1,000 to 10,000 values, 10,000 to 100,000 values, or more than 100,000 values. Synthetic event 202 is also described in US provisional application corresponding to docket number 109304-793421-008200USP, the entire contents of which are incorporated herein by reference for all purposes.

Synthetic event 202 is analyzed by event processor 204. Event processor 204 may clean up data. Event processor 204 may filter out noise events to reduce the chance of false positives. Event processor 204 may access data not related to sensors for user 102. For example, other data may include user historical data, environmental data (e.g., pollen/allergy), medications being taken, location-based data (e.g., weather or proximity to environmental event [e.g., toxic material, chemical spill]). Event processor 204 may include data normalization, vector-based matching against training data, and/or adjusting/fine-tuning custom parameters. Vector-based matching may determine how close the vector or matrix of the synthetic event is similar to reference data. Event processor 204 may use a machine learning model, which may be trained using reference synthetic event data and filtered synthetic event data. The machine learning model may have synthetic event data as an input and then return filtered/processed synthetic event data. Machine learning architecture 600 may be used for any machine learning model described herein.

Event classifier 206 assigns one or more synthetic events 202 to a classification. The classifying may use categories and/or ranges of synthetic events 202. For example, synthetic events related to heart rate, blood pressure, and body temperature may be grouped together as relevant for determining a heart attack, stroke, or other disorder. These synthetic events may need to be within or outside certain ranges to be classified as relevant for some life event. Vector-based matching may be used to compare vectors or matrices of multiple synthetic events to reference data to see if a classification applies. Event classifier 206 may be a machine learning model, which may be trained using reference synthetic event data and event classifications. The machine learning model may have synthetic event data as an input and then output the event classification. Event classifier 206 and event processor 204 may occur in parallel to expedite analysis.

Anomaly detector 208 compares the synthetic event data for the classification with data for the classification from model baseline 214. Anomaly detector 208 determines if the event is out of the ordinary for the category. Anomaly detector 208 may include cloud anomaly detector 122 and/or on-site anomaly detector 124. An anomaly may be detected if the synthetic event data is significantly different from model baseline 214. The detection of an anomaly may not be a comparison of a single value with a threshold value. Instead, the detection of an anomaly may involve comparing vectors or matrices of one or more synthetic events with model baseline 214. An anomaly may be detected even if not all values of vectors or matrices of the synthetic events exceed the respective thresholds. Anomaly detector 208 may be a machine learning model. The machine learning model may be trained using reference synthetic event data and anomaly classifications (e.g., present or not present). The machine learning model may have synthetic event data as an input and then output whether an anomaly is present. In some embodiments, an anomaly may be detected using natural language processing or a large language model. The collection of synthetic events may be analogous to words in a sentence. These words can then be used to detect an anomaly or classify the absence of an anomaly. If an anomaly is detected, a notification event 212 may be generated. Notification event 212 is described in more detail with FIG. 3.

A synthetic event 202 may lead to model augmentation 210. The data for the event may be used to further train a machine learning model or may be used to update model baseline 214. If a notification event 212 is generated and determined by user feedback or other means to be a accurate notification, then the anomaly detector 208 can be trained with this information. If a notification event 212 is generated and determined by user feedback or other means to be an inaccurate notification, the anomaly detector 208 can be trained with this information. A human may also augment the model based on anomaly detection and notifications. Model augmentation 210 may result in a new model baseline 214. Model augmentation 210 may continue with more anomalies detected or considered as not present.

FIG. 3 shows an overview of a notification system 300 for a notification event 302. Notification event 302 may include notification event 212 detected by anomaly detector 208. Notification events may be classified based on category and degree of severity. Based on the classification, the event may need to be broadcast and handled by observers like other end-users, caregivers, or professionals.

A notification event 302 may be categorized using an anomaly detection mechanism as described with FIG. 2. Each event may be assigned a category and range. Both heuristic and probabilistic methods may be used to determine whether the event merits notification and/or escalation.

Notification event 302 may be broadcast at 304. Broadcast 304 may be performed using a publisher/subscriber model, where the same event may be used to notify multiple observers in parallel or through an escalation list.

Notification event 302 may be broadcast to individual 330, the person experiencing the synthetic event. The notification may be routed to a gateway 306. Gateway 306 may be a mobile device (e.g., cell phone or tablet) or may be a standalone and/or dedicated device for monitoring and alerting individual 330. Gateway 306 may display a user interface 314. User interface 314 may notify individual 330.

Notification event 302 may be sent to monitor service 308. Monitor service 308 may be used in shared facilities (e.g., nursing homes). Monitor service 308 may be a central monitoring service where trained personnel can take action depending on the severity of the event. Monitor service 308 may notify trained personnel using monitor network 316. Monitor network 316 may include a central display or devices distributed to personnel.

Notification event 302 may be broadcast to mobile app 310. Mobile app 310 may notify people designated as contacts for individual 330, such as first contact 318 and/or second contact 320. First contact 318 and second contact 320 may be relatives, friends, or other people who may be able to make decisions on care of individual 330. First contact 318 and/or second contact 320 may have the option in mobile app 310 to communicate with individual 330.

Notification event 302 may involve a workflow trigger 312. Workflow trigger 312 may wait for individual 330, caregivers, or contacts to perform an action (e.g., acknowledge an alert, contacting the individual 330). In case, no action is performed within a time limit, then the notification system 300 may escalate the event to external services such as medical providers or emergency services. The time limit may vary by the severity of the event.

The raw data stream that induced the event may be saved so that the data stream can be observed and audited by caregivers, medical personnel, and emergency services. The raw data stream can also be used to perform root cause analysis of events to prevent a recurrence.

The workflow trigger may be based on an adjustable “threshold” level. This may be a numeric value that is computed based on the severity of the event and the urgency with which the event has to be handled. A highly “critical” event may trigger all caregivers, medical personnel 326, and emergency services 328 simultaneously. A less critical one may trigger a workflow that notifies the primary caregiver, then waits a predetermined length of time for acknowledgement before escalating to the next caregiver. As an example, a heart attack may be a highly critical event, which may result in a notification to all caregivers, medial medical personnel 326, and emergency services 328. For example, a fall event may have a shorter time limit than an event indicating individual 330 is lost.

Medical personnel 326 concerned about a patient's well-being may adjust the threshold for notification so they are informed more quickly if individual 330 triggers an event.

The workflow for a notification event 212 may use a machine learning model. The machine learning model may be trained using reference notification events and information regarding a notification workflow. The information of the notification workflow may include whether such notifications were effective or acknowledged. For example, a notification of first contact 318 may have been ignored with certain notification events 212. The machine learning model can be trained to adjust the workflow to skip notification of first contact 318 in certain situations.

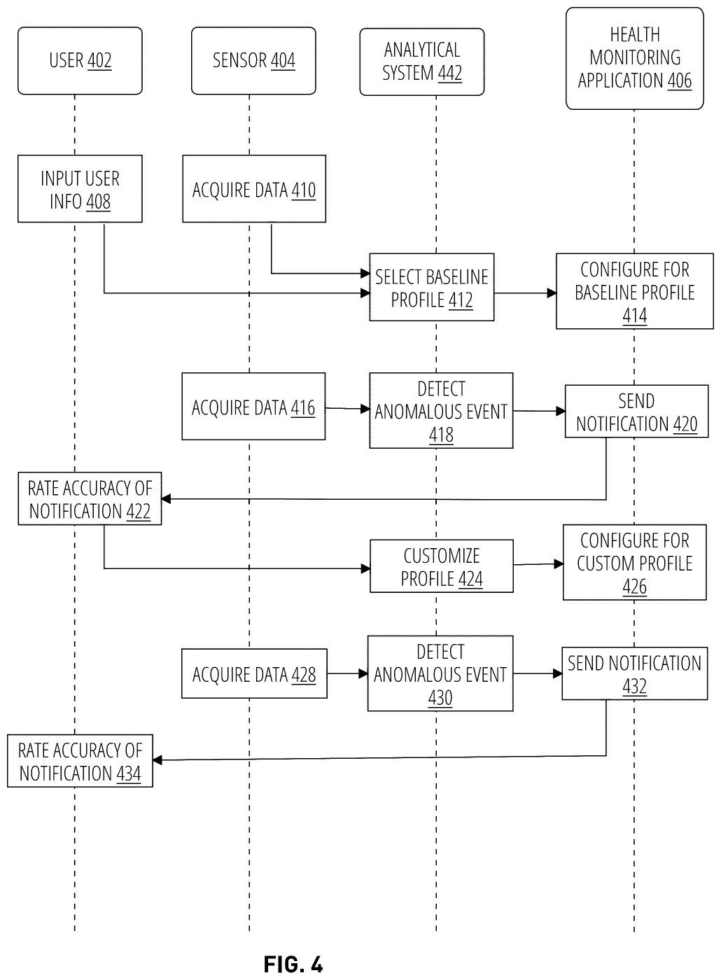

FIG. 4 illustrates the operations performed by various components. User 402 is the subject of the monitoring. Sensor 404 may be any sensor described herein. Sensor 404 may be part of a mobile device (e.g., a cellular phone, smartwatch). For example, sensor 404 may be an accelerometer, a global positioning system (GPS) sensor, or a microphone on a phone. In some embodiments, sensor 404 may be a heart rate monitor or a blood oxygen monitor (e.g., on a smartwatch). In embodiments, sensor 404 may be device not in contact with user 402. For example, sensor 404 may include Internet-of-Things (IoT) devices including motion sensors, thermostats, light switches, or weighing scales. Sensor 404 may also include environmental sensors, such as microphones, video cameras, mmWave motion detectors, or on-device fall detection sensors.

Analytical system 442 may include a computing system equipped to receive and send data and to perform calculations on the data. Analytical system 442 may be a cloud-based system, an on-site system (e.g., gateway 306), a mobile device system, a wearable device, an in-body embedded device, or a combination thereof. Analytical system 442 may house a machine learning model.

Health monitoring application 406 may be an application that communicates to user 402, another individual (first contact 318 or second contact 320), or another system (e.g., monitor service 308). Health monitoring application 406 may reside on a mobile device, a cloud-based system, an on-site system, a wearable device, an in-body embedded device, or a combination thereof. In some embodiments, health monitoring application 406 may include analytical system 442.

User 402 may input user information 408 into analytical system 442. The user info may include user attributes 104 or any attribute described herein. The user may input through a user interface on their mobile device or computer. In some embodiments, another individual (e.g., a relative or caregiver) may input the user information.

Sensor 404 may acquire data 410 related to user 402 over a period of time. The data may include location, activity, heart rate, blood oxygen, temperature, weight, or other data. The data may be sent to analytical system 442 through suitable communication techniques.

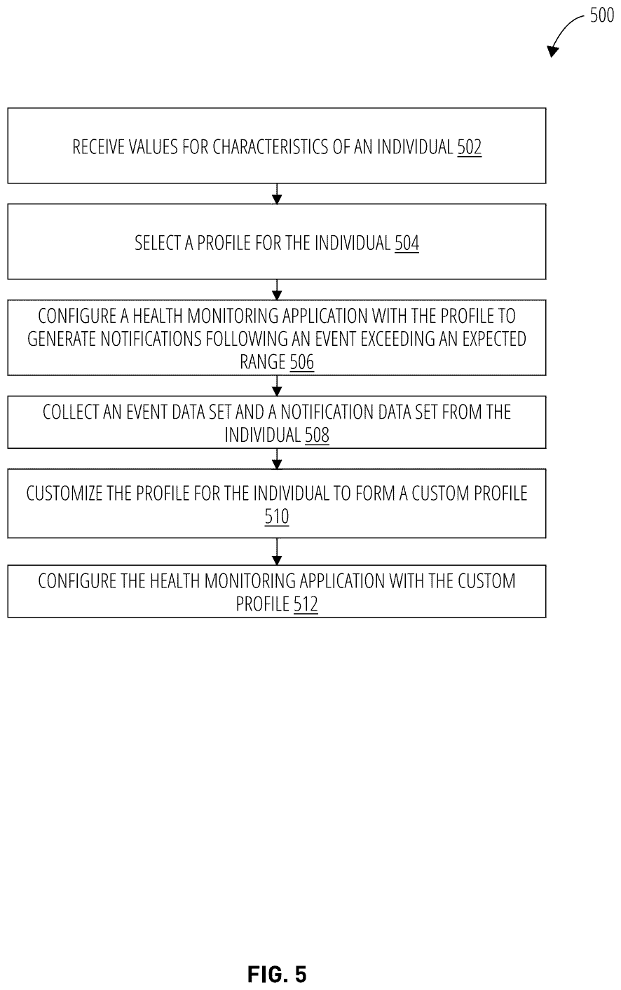

Analytical system 442 may analyze the inputted user information and sensor data to select baseline profile 412. Analytical system 442 may include model generator 118. Analytical system 442 may compare user input information and sensor data with analogous information associated with a set of profiles. Analytical system 442 may then select a baseline profile, which may be the profile that is most similar to user input information and/or sensor data. The selection of the baseline profile is described in more detail with FIG. 2 and FIG. 5.

Health monitoring application 406 is configured for the selected baseline profile 414. Configuring the health monitoring application 406 may involve saving the baseline profile to memory.

After the health monitoring application 406 is configured for the baseline profile 414, sensor 404 may acquire data 416. The additional sensor data may be compared to the baseline profile by analytical system 442. In some embodiments, the comparison may be performed by cloud anomaly detector 122, an on-site anomaly detector 124, or anomaly detector 208. Analytical system 442 may detect an anomalous event 418. The anomalous event may be an event that is outside some confidence range for expected events given the baseline profile. The detection of the anomalous event is described in more detail with FIG. 2 and FIG. 5. Detection of the anomalous event may include any steps involved with event processor 204, event classifier 206, and anomaly detector 208.

Health monitoring application 406 receives an alert regarding an anomalous event from analytical system 442. Health monitoring application 406 sends a notification 420 of the anomalous event to user 402, another individual, and/or another system. The notification may be an alert in the application on a mobile device, a watch, a custom wearable, an alert on a standalone system (e.g., gateway 306), a text message, an email, a phone call, a sound, a graphic, a light, an electronic notification, or any other notification. The alert may be on a device with a siren, display, light, or other indicator. The device may be used to monitor multiple people. The notification may be any notification described herein, including with notification event 212 and notification event 302. To send the notification, analytical system 442 or health monitoring application 406 may determine notification recipients, the triggering event workflow (e.g., which paths of FIG. 3 to take), and/or the mode of notification.

User 402 may receive the notification and rate the accuracy of the notification 422 through user feedback. For example, user 402 may dismiss the notification or ignore the notification without taking any action. As another example, user 402 may enter a rating for the notification using health monitoring application 406. The rating may be a numerical rating (e.g., a scale of 1 to 10) or a categorical rating (e.g., not useful, not accurate, invalid). FIG. 4 shows the rating of the notification by user 402 for illustrative purposes. Rating the accuracy may be performed by individuals other than user 402 (e.g., contacts or health care professionals).

In some embodiments, between send notification 420 and rate accuracy of notification 422, analytical system 442 may have timeout mechanism to ascertain whether a caregiver or user 402 has responded in time and/or whether the event was escalated or handled appropriately, similar to what was discussed with event handled 322 and event timeout 324.

The notification accuracy rating may be sent to analytical system 442. The analytical system 442 analyzes the accuracy ratings. If a sufficient number of low notification accuracy ratings are received, then analytical system 442 may customize the user profile 424. In some embodiments, a notification may be associated with a severe event (e.g., a heart attack). A single poor accuracy rating on a severe event may be sufficient to result in the analytical system 442 customizing the profile 424. A sliding scale of number of low notification accuracy ratings and severity of the event may be used to determine whether to customize the profile. For example, a smaller number of low notification accuracy ratings may be sufficient if the severity of the event is higher than average. The profile may be customized by a machine learning model. The customization is described in greater detail in FIG. 5.

Health monitoring application 406 may be configured for the custom profile 426, replacing the baseline profile. Sensor 404 may acquire data 428. The data may be analyzed by analytical system 442 in comparison with the custom profile. When the event exceeds the range of expected events for the custom profile, then the analytical system 442 detects an anomalous event 430. The health monitoring application 406 is alerted of the anomalous event. Health monitoring application 406 sends a notification 432 of the anomalous event, as described with send notification 420.

User 402 may rate the accuracy of notification 434, similar to rate accuracy of notification 422. Because user 402 now has a customized profile based on specific user data, notifications will have a higher accuracy (lower false positive rate) than with the baseline profile. In some embodiments, there may still be too many false notifications. Another custom profile may be generated, and the steps may repeat until the false notifications are sufficiently reduced.

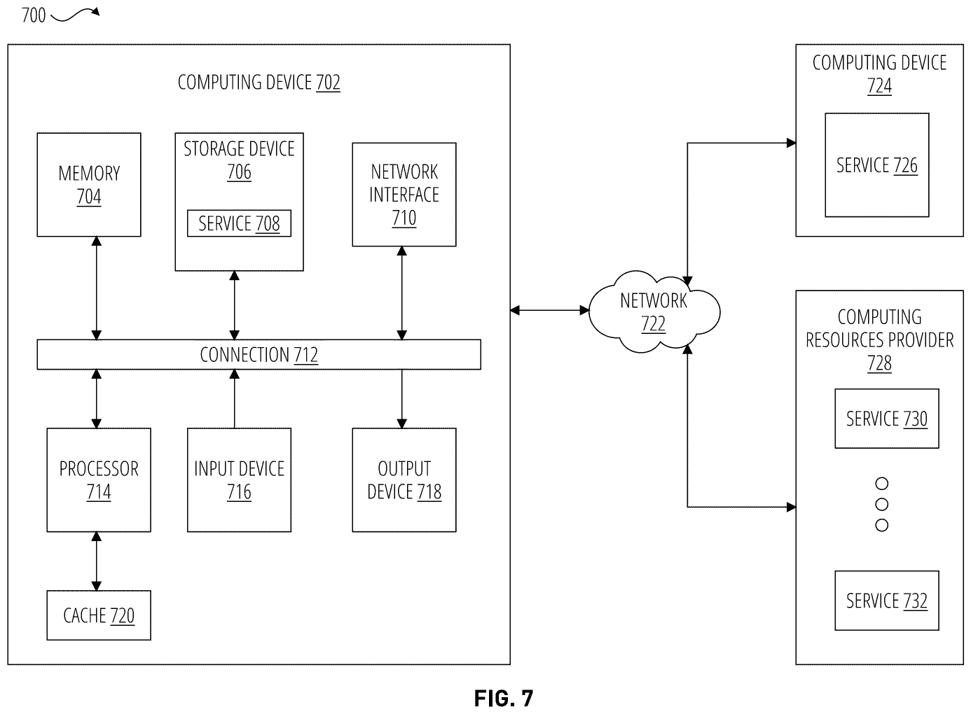

FIG. 5 shows a method 500 for monitoring the health of an individual. Method 500 may be implemented by any components of FIG. 1 to FIG. 4, FIG. 6, and FIG. 7 or other systems described herein.

At block 502, method 500 receives values for characteristics of an individual. The characteristics may include physical properties or medical history properties of the individual. The characteristic may be a trendline of a physical property or medical property. The characteristics may include user attributes 104. The values of the characteristics may be received as user input 408 and/or sensor 404 data.

At block 504, method 500 selects a profile for the individual. Selecting may include using the values of the characteristics. The profile may specify expected ranges for event data. Expected ranges may include a minimum or maximum for the value of the characteristic, ramps, the first derivative of the characteristic, and/or the second derivative of the characteristic. Selecting the profile may include determining similarities between the individual and a plurality of profiles using the values of the characteristics. Selecting the profile may include identifying the profile as having the highest similarity with the individual among the plurality of profiles, as described with model generator 118, user attributes 104, and model baseline 214.

In some embodiments, selecting the profile may include simulating anomalous event detection with any event data from the individual against the plurality of profiles. The profile with the fewest false anomalous detections may be selected. The profile with a sufficient threshold of accuracy (e.g., sensitivity, specificity) may be selected.

In some embodiments, selecting the profile may include generating the profile from a plurality of profiles using the values of the characteristics. For example, the profile may be an average or a weighted average of the plurality of profiles. The weights may be based on how similar a profile is to the individual (e.g., a more similar profile may have a higher weight).

At block 506, method 500 configures a health monitoring application with the profile to generate notifications following an event exceeding an expected range. The health monitoring application may be health monitoring application 406 or any application described herein. The expected range may be a window around an average value for the data. For example, the window may be one, two, or three standard deviations around the average. The window may be a certain confidence limit or percentile around the average. For example, the health monitoring application may within 5%, 10%, 15%, 20%, 30%, 40%, or 50% of the confidence limit or percentile around the average. The profile may include several ranges for several different characteristics. For example, the profile may include a range for a first derivative of a first characteristic, a range for a median value of the first characteristic, and a range for a first derivative of a second characteristic.

At block 508, method 500 collects an event data set and a notification data set from the individual. Event data may include raw data such as heartrate data, step data, sleep data, activity data, appliance usage data, dietary data, medication data, location data, accelerometer data, blood pressure data, body temperature data, ambient temperature data, blood sugar data, sound data, or other sensor data. Sensor data may be acquired by sensor 404 or any sensor described herein. The event data may be sensor data but may also be determined using a combination of different sensor data. For example, an event such as a stroke may be determined from different types of sensor data, including heartrate, acceleration, location, body temperature, speech patterns, and/or speech volume. In some embodiments, the event may be a synthetic event, which is an event not necessarily tied to any specific condition. Rather, a synthetic event may have been determined to coincide with certain health issues.

At block 510, method 500 customizes the profile for the individual to form a custom profile. Customizing may include using a trained machine learning model to process a data set to output accurate notifications. The data set may include the event data set, the notification data set, and a set of labels indicating the accuracy of notifications in the notification data set. Customizing the profile may be similar to model augmentation 210.

Method 500 may include training the machine learning model. The machine learning model may be trained on the data set. The data set may be acquired from sensors associated with the individual. In some embodiments, the data set may include data from other individuals, including reference individual(s). The reference individual(s) may include the individual(s) associated with the baseline profile. Parameters of the machine learning model may be optimized so that the output of the machine learning model matches the label indicating the accuracy of the notification of an anomalous event. For example, the machine learning model may calculate a probability that an anomalous event has occurred. The machine learning model may adjust the threshold probability required to output notification of the anomalous event.

Customizing the profile may be preceded by calculating an accuracy score for notifications generated by the selected baseline profile from the event data set. Method 500 may include determining the accuracy score is less than a threshold value. The determination of the accuracy score being less than the threshold value may trigger block 510. The accuracy score may be a false positive rate, a true positive rate, a false negative rate, a true negative rate, a sensitivity, a specificity, an area under the curve (AUC) for a receiver operating characteristic (ROC) curve, or another statistical measure of accuracy. The threshold value of accuracy may be predetermined or set or adjusted by the individual.

At block 512, method 500 configures the health monitoring application with the custom profile. Configuring the health monitoring application with the custom profile may include calculating an accuracy score for notifications generated by the custom profile using the event data set. The accuracy score may be the same score as the accuracy score for the selected baseline profile. The accuracy score may be determined to be greater than a threshold value. The threshold value may be the same threshold value used for evaluating the previously selected profile. In some embodiments, the accuracy score of the previously selected profile may be the threshold value for the custom profile.

Method 500 may include detecting an anomalous event using the health monitoring application configured with the custom profile. Detecting the anomalous event may include collecting a second event data set. A first portion of the second data set may be analyzed to determine a first baseline. In some embodiments, the first portion of the second event data set may be the entirety or less than the entirety of the second event data set. A second portion of the second event data may be analyzed to determine a second baseline. The second portion of the second event data set may or may not overlap with the first portion. The second portion may include more recent data than the first portion. The first baseline may be compared to the second baseline.

Method 500 may include determining that no baseline shift has occurred based on the comparison. Determining that no baseline shift has occurred may be performed using a statistical test. Statistical tests may include the Two-Sample T-Test, Paired T-Test, Z-Test, Mann-Whitney U Test, Wilcoxon Signed-Rank Test, Kolmogorov-Smirnov Test, Levene's Test, Bartlett's Test, Cumulative Sum Control Chart (CUSUM), Exponentially Weighted Moving Average (EWMA), Hotelling's T-Squared Test, Measure of Scale (MOS), Chi-Square Test, F-Test, Anderson-Darling, Grubbs Test, measuring kurtosis and skew, or Tietjen-Moore Test for Outliers. For no baseline shift, the statistical test may indicate no statistical difference between the first portion and the second portion.

The detected anomalous event may be classified by generating a severity score for the anomalous event. The severity score may be generated using clinical scores, individual feedback, or suitable grading scales. In some embodiments, the anomalous event may be sent through a diagnostics generative large language model (LLM) to identify severity. The data of the event may be the words used in the LLM, and the presence of the anomalous event and/or the severity may be an output of the LLM. A higher severity score may require certain types of notifications.

Method 500 may include sending a notification of the anomalous event. Sending notification of the anomalous event may include sending a communication to the individual, to a service monitoring the individual, or a contact designated by the individual. The notification may be any notification described with FIG. 3 or FIG. 4. Method 500 may include detecting whether an acknowledgment of the communication is received (e.g., as described with workflow trigger 312). Method 500 may include calling a medical practitioner upon not detecting the acknowledgment within a time window. A high severity score may result in a notification to the medical practitioner and/or a shorter time window for an acknowledgment.

In some embodiments, method 500 may include detecting an anomalous event using the health monitoring application configured with the custom profile. Detecting the anomalous event may include collecting a second event data set. A first portion of the second data set may be analyzed to determine a first baseline. A second portion of the second event data may be analyzed to determine a second baseline. The first baseline may be compared to the second baseline, as described with determining no baseline shift. Method 500 may include determining that a baseline shift has occurred based on the comparison. For example, the statistical test may indicate that the first portion is statistically different from the second portion. The individual may be prompted to confirm the baseline shift. The baseline shift may include a significant life event or a stressful event (e.g., a surgery, a illness [including one to a close contact], a change in occupation, a relocation, the start of treatment, the end of treatment). In some embodiments, a notification may be sent that the anomalous event is a result of the baseline shift. In embodiments, no notification may be sent as a result of the anomalous event. In some embodiments, the baseline shift may be determined before the anomalous event detection.

In some embodiments, a disease or disorder may be detected as the anomalous event. A notification of the disease or disorder may be sent to medical personnel. The individual may be treated for the disease or disorder. Examples of such treatment can include surgery, radiation therapy, chemotherapy, immunotherapy, targeted therapy, hormone therapy, stem cell transplant, drugs, or diet. The disease or disorder may include a cardiovascular disease, arthritis, osteoporosis, diabetes, a respiratory disease, a neurological disorder, a cancer, a stroke, a vision and/or hearing problem, a mental health issue, a gastrointestinal issue, a urinary disorder, and a skin condition.

Embodiments may include a computer-readable medium upon which computer-executable instructions are stored to implement method 500. Embodiments may include a computing system. The computing system may include a processor and a memory storing instructions configured such that, when executed in cooperation with controlling the processor, the instructions operate the computing system to perform method 500.

Embodiments may include a system. The system may include the computing system and a treatment device, which can provide a treatment to the individual. The treatment device can determine a treatment and/or be used to perform a treatment. The treatment device may receive inputs from other devices, such as an imaging device and user inputs (e.g., to control the treatment, such as controls over a robotic system).

FIG. 6 is a block diagram of illustrating a machine learning architecture 600 using one or more machine learning model(s) 608 of a machine learning engine 606 to analyze data to produce an output. Examples of the machine learning model(s) 608 include any machine learning model described herein. The machine learning engine 606 generates, trains, and uses the machine learning model(s) 608 based on an initial training using training data 604. The machine learning engine 606 trains the machine learning model(s) 608 to generate an analysis on input of sample data 602 into the machine learning model(s) 608. The sample data 602 may include data that is stored on a data source.

The analysis output by the machine learning model(s) 608 can include at least one output identified as part of the analysis of the sample data. The output can be any type of output, for instance including outputs associated with event processors, event classifiers, anomaly detectors, model augmentation, and/or notifications. In some examples, the analysis output by the machine learning model(s) 608 can include various other elements of analyses described herein as output by any system described herein. For instance, the analysis output by the machine learning model(s) 608 can include filtered synthetic event data, event classifications, anomaly detections, notifications, or combinations thereof. The analysis can include a confidence score. The confidence score can be compared to a threshold. A confidence score above the threshold may result in determining that a certain classification/anomaly exists.

The training data 604 that the machine learning engine 606 uses to train the machine learning model(s) 608 may include sample data (e.g., akin to the sample data 602) as well as pre-generated assessment(s) corresponding to the sample data (e.g., akin to the analysis based on sample data 610). Over the course of the initial training, the machine learning model(s) 608 develop hidden layers between input layers and output layers, and/or weights and/or connections between nodes of the various layers, that each relate to various aspects of the analysis based on sample data 610, such as any of the aspects described herein (e.g., related to various types of outputs and characteristics of those types of outputs). Training of the machine learning model may include optimizing parameters (e.g., hidden parameters) of the model so as to increase accuracy (e.g., reduce cost function).

Once the one or more machine learning model(s) 608 identify the analysis 610, the analysis 610 can be output to a user and/or to a device described herein.

In some examples, the machine learning engine 606 can continue to train and/or update the machine learning model(s) 608 over time, for instance based on validation 612 using the analysis 610 and the sample data 602. In some examples, an analysis of the sample data 602 (separate from the analysis 610 generated by the machine learning model(s) 608) may be provided to the machine learning engine 606 in performing the validation 612. In some examples, the analysis may be generated by a different entity than the ML machine learning model(s) 608, for instance a different set of ML model(s) (not pictured) or one or more trusted human analysts. If, during validation 612, the machine learning engine 606 determines that the analysis 610 generated by the machine learning model(s) 608 matches the analysis of the sample data 602, the machine learning engine 606 can treat this as positive feedback, and can perform further training of the ML machine learning model(s) 608 based on the analysis 610, the sample data 602, and/or the analysis of the sample data 602, for instance to strengthen and/or reinforce weights associated with generating the analysis 610 in the machine learning model(s) 608, and/or to weaken or remove other weights other than those associated with generating the analysis 610, in the machine learning model(s) 608. If, during validation 612, the machine learning engine 606 determines that the 610 generated by the machine learning model(s) 608 differs from the analysis of sample data 602, the machine learning engine 606 can treat this as negative feedback, and can perform additional training 614 of the ML machine learning model(s) 608 based on the analysis 610, the sample data 602, and/or the analysis of the sample data 602, for instance to weaken and/or remove weights associated with generating the analysis 610 in the machine learning model(s) 608, and/or to strengthen and/or reinforce other weights other than those associated with generating the analysis 610 in the machine learning model(s) 608.

In some examples, the machine learning engine 606 receives feedback about the analysis 610. The feedback can include a reaction by a user of a user device via a user interface, a reaction by a user determined based on sensor data from a user device, and/or decisions by a user and/or user device as whether or not to use the analysis 610 for a further application. Positive feedback can be used to strengthen and/or reinforce weights associated with generating the analysis 610 in the machine learning model(s) 608, and/or to weaken or remove other weights other than those associated with generating the analysis 610 in the machine learning model(s) 608. Negative feedback can be used to weaken and/or remove weights associated with generating the analysis 610 in the machine learning model(s) 608, and/or to strengthen and/or reinforce other weights other than those associated with generating the analysis 610 in the machine learning model(s) 608.

Machine learning architecture 600 may be used with any of the methods and systems described herein.

FIG. 7 illustrates a computing system architecture 700, including various components in electrical communication with each other, in accordance with some embodiments. The example computing system architecture 700 illustrated in FIG. 7 includes a computing device 702, which has various components in electrical communication with each other using a connection 712, such as a bus, in accordance with some implementations. The example computing system architecture 700 includes a processor 714 that is in electrical communication with various system components, using the connection 712, and including the system memory 704. In some embodiments, the system memory 704 includes read-only memory (ROM), random-access memory (RAM), and other such memory technologies including, but not limited to, those described herein. In some embodiments, the example computing system architecture 700 includes a cache 720 of high-speed memory connected directly with, in close proximity to, or integrated as part of the processor 714. The computing system architecture 700 can copy data from the memory 704 and/or the storage device 706 to the cache 720 for quick access by the processor 714. In this way, the cache 720 can provide a performance boost that decreases or eliminates processor delays in the processor 714 due to waiting for data. Using modules, methods and services such as those described herein, the processor 714 can be configured to perform various actions. In some embodiments, the cache 720 may include multiple types of cache including, for example, level one (L1) and level two (L2) cache. The memory 704 may be referred to herein as system memory or computer system memory. The memory 704 may include, at various times, elements of an operating system, one or more applications, data associated with the operating system or the one or more applications, or other such data associated with the computing device 702.

Other system memory 704 can be available for use as well. The memory 704 can include multiple different types of memory with different performance characteristics. The processor 714 can include any general purpose processor and one or more hardware or software services, such as service 708 stored in storage device 706, configured to control the processor 714 as well as a special-purpose processor where software instructions are incorporated into the actual processor design. The processor 714 can be a completely self-contained computing system, containing multiple cores or processors, connectors (e.g., buses), memory, memory controllers, caches, etc. In some embodiments, such a self-contained computing system with multiple cores is symmetric. In some embodiments, such a self-contained computing system with multiple cores is asymmetric. In some embodiments, the processor 714 can be a microprocessor, a microcontroller, a digital signal processor (“DSP”), or a combination of these and/or other types of processors. In some embodiments, the processor 714 can include multiple elements such as a core, one or more registers, and one or more processing units such as an arithmetic logic unit (ALU), a floating point unit (FPU), a graphics processing unit (GPU), a physics processing unit (PPU), a digital system processing (DSP) unit, or combinations of these and/or other such processing units.

To enable user interaction with the computing system architecture 700, an input device 716 can represent any number of input mechanisms, such as a microphone for speech, a touch-sensitive screen for gesture or graphical input, keyboard, mouse, motion input, pen, and other such input devices. An output device 718 can also be one or more of a number of output mechanisms known to those of skill in the art including, but not limited to, monitors, speakers, printers, haptic devices, and other such output devices. In some instances, multimodal systems can enable a user to provide multiple types of input to communicate with the computing system architecture 700. In some embodiments, the input device 716 and/or the output device 718 can be coupled to the computing device 702 using a remote connection device such as, for example, a communication interface such as the network interface 710 described herein. In such embodiments, the communication interface can govern and manage the input and output received from the attached input device 716 and/or output device 718. As may be contemplated, there is no restriction on operating on any particular hardware arrangement and accordingly the basic features here may easily be substituted for other hardware, software, or firmware arrangements as they are developed.

In some embodiments, the storage device 706 can be described as non-volatile storage or non-volatile memory. Such non-volatile memory or non-volatile storage can be a hard disk or other types of computer readable media which can store data that are accessible by a computer, such as magnetic cassettes, flash memory cards, solid state memory devices, digital versatile disks, cartridges, RAM, ROM, and hybrids thereof.

As described above, the storage device 706 can include hardware and/or software services such as service 708 that can control or configure the processor 714 to perform one or more functions including, but not limited to, the methods, processes, functions, systems, and services described herein in various embodiments. In some embodiments, the hardware or software services can be implemented as modules. As illustrated in example computing system architecture 700, the storage device 706 can be connected to other parts of the computing device 702 using the system connection 712. In some embodiments, a hardware service or hardware module such as service 708, that performs a function can include a software component stored in a non-transitory computer-readable medium that, in connection with the necessary hardware components, such as the processor 714, connection 712, cache 720, storage device 706, memory 704, input device 716, output device 718, and so forth, can carry out the functions such as those described herein.

The disclosed processed for generating and executing experience recommendations can be performed using a computing system such as the example computing system illustrated in FIG. 7, using one or more components of the example computing system architecture 700. An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device.

In some embodiments, the processor can be configured to carry out some or all of methods and functions for generating and executing experience recommendations described herein by, for example, executing code using a processor such as processor 714 where the code is stored in memory such as memory 704 as described herein. One or more of a user device, a provider server or system, a database system, or other such devices, services, or systems may include some or all of the components of the computing system such as the example computing system illustrated in FIG. 7, using one or more components of the example computing system architecture 700 illustrated herein. As may be contemplated, variations on such systems can be considered as within the scope of the present disclosure.

This disclosure contemplates the computer system taking any suitable physical form. As example and not by way of limitation, the computer system can be an embedded computer system, a system-on-chip (SOC), a single-board computer system (SBC) (such as, for example, a computer-on-module (COM) or system-on-module (SOM)), a desktop computer system, a laptop or notebook computer system, a tablet computer system, a wearable computer system or interface, an interactive kiosk, a mainframe, a mesh of computer systems, a mobile telephone, a personal digital assistant (PDA), a server, or a combination of two or more of these. Where appropriate, the computer system may include one or more computer systems, be unitary or distributed, span multiple locations; span multiple machines; and/or reside in a cloud computing system which may include one or more cloud components in one or more networks as described herein in association with the computing resources provider 728. Where appropriate, one or more computer systems may perform without substantial spatial or temporal limitation one or more steps of one or more methods described or illustrated herein. As an example and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor 714 can be a conventional microprocessor such as an Intel® microprocessor, an AMD® microprocessor, a Motorola® microprocessor, a Qualcomm® microprocessor, an Apple® microprocessor, or other such microprocessors. One of skill in the relevant art will recognize that the terms “machine-readable (storage) medium” or “computer-readable (storage) medium” include any type of device that is accessible by the processor. In some embodiments, processor 714 may be an application-specific integrated circuit (ASIC).

The memory 704 can be coupled to the processor 714 by, for example, a connector such as connection 712, or a bus. As used herein, a connector or bus such as connection 712 is a communications system that transfers data between components within the computing device 702 and may, in some embodiments, be used to transfer data between computing devices. The connection 712 can be a data bus, a memory bus, a system bus, or other such data transfer mechanism. Examples of such connectors include, but are not limited to, an industry standard architecture (ISA bus, an extended ISA (EISA) bus, a parallel AT attachment (PATA bus (e.g., an integrated drive electronics (IDE) or an extended IDE (EIDE) bus), or the various types of parallel component interconnect (PCI) buses (e.g., PCI, PCIe, PCI-104, etc.).

The memory 704 can include RAM including, but not limited to, dynamic RAM (DRAM), static RAM (SRAM), synchronous dynamic RAM (SDRAM), non-volatile random access memory (NVRAM), and other types of RAM. The DRAM may include error-correcting code (EEC). The memory can also include ROM including, but not limited to, programmable ROM (PROM), erasable and programmable ROM (EPROM), electronically erasable and programmable ROM (EEPROM), Flash Memory, masked ROM (MROM), and other types or ROM. The memory 704 can also include magnetic or optical data storage media including read-only (e.g., CD ROM and DVD ROM) or otherwise (e.g., CD or DVD). The memory can be local, remote, or distributed.

As described above, the connection 712 (or bus) can also couple the processor 714 to the storage device 706, which may include non-volatile memory or storage and which may also include a drive unit. In some embodiments, the non-volatile memory or storage is a magnetic floppy or hard disk, a magnetic-optical disk, an optical disk, a ROM (e.g., a CD-ROM, DVD-ROM, EPROM, or EEPROM), a magnetic or optical card, or another form of storage for data. Some of this data is may be written, by a direct memory access process, into memory during execution of software in a computer system. The non-volatile memory or storage can be local, remote, or distributed. In some embodiments, the non-volatile memory or storage is optional. As may be contemplated, a computing system can be created with all applicable data available in memory. A typical computer system will usually include at least one processor, memory, and a device (e.g., a bus) coupling the memory to the processor.

Software and/or data associated with software can be stored in the non-volatile memory and/or the drive unit. In some embodiments (e.g., for large programs) it may not be possible to store the entire program and/or data in the memory at any one time. In such embodiments, the program and/or data can be moved in and out of memory from, for example, an additional storage device such as storage device 706. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The connection 712 can also couple the processor 714 to a network interface device such as the network interface 710. The interface can include one or more of a modem or other such network interfaces including, but not limited to those described herein. It will be appreciated that the network interface 710 may be considered to be part of the computing device 702 or may be separate from the computing device 702. The network interface 710 can include one or more of an analog modem, Integrated Services Digital Network (ISDN) modem, cable modem, token ring interface, satellite transmission interface, or other interfaces for coupling a computer system to other computer systems. In some embodiments, the network interface 710 can include one or more input and/or output (I/O) devices. The I/O devices can include, by way of example but not limitation, input devices such as input device 716 and/or output devices such as output device 718. For example, the network interface 710 may include a keyboard, a mouse, a printer, a scanner, a display device, and other such components. Other examples of input devices and output devices are described herein. In some embodiments, a communication interface device can be implemented as a complete and separate computing device.

In some embodiments, the computing device 702 can be connected to one or more additional computing devices such as computing device 724 via a network 722 using a connection such as the network interface 710. In such embodiments, the computing device 724 may execute one or more services 726 to perform one or more functions under the control of, or on behalf of, programs and/or services operating on computing device 702. In some embodiments, a computing device such as computing device 724 may include one or more of the types of components as described in connection with computing device 702 including, but not limited to, a processor such as processor 714, a connection such as connection 712, a cache such as cache 720, a storage device such as storage device 706, memory such as memory 704, an input device such as input device 716, and an output device such as output device 718. In such embodiments, the computing device 724 can carry out the functions such as those described herein in connection with computing device 702. In some embodiments, the computing device 702 can be connected to a plurality of computing devices such as computing device 724, each of which may also be connected to a plurality of computing devices such as computing device 724. Such an embodiment may be referred to herein as a distributed computing environment.

In some embodiments, the computing device 702 and/or the computing device 724 can be connected to a computing resources provider 728 via the network 722 using a network interface such as those described herein (e.g. network interface 710). In such embodiments, one or more systems (e.g., service 730 and service 732) hosted within the computing resources provider 728 (also referred to herein as within “a computing resources provider environment”) may execute one or more services to perform one or more functions under the control of, or on behalf of, programs and/or services operating on computing device 702 and/or computing device 724. Systems such as service 730 and service 732 may include one or more computing devices such as those described herein to execute computer code to perform the one or more functions under the control of, or on behalf of, programs and/or services operating on computing device 702 and/or computing device 724.

For example, the computing resources provider 728 may provide a service, operating on service 730 to store data for the computing device 702 when, for example, the amount of data that the computing device 702 exceeds the capacity of storage device 706. In another example, the computing resources provider 728 may provide a service to first instantiate a virtual machine (VM) on service 732, use that VM to access the data stored on service 732, perform one or more operations on that data, and provide a result of those one or more operations to the computing device 702. Such operations (e.g., data storage and VM instantiation) may be referred to herein as operating “in the cloud,” “within a cloud computing environment,” or “within a hosted virtual machine environment,” and the computing resources provider 728 may also be referred to herein as “the cloud.” Examples of such computing resources providers include, but are not limited to Amazon® Web Services (AWS®), Microsoft's Azure®, IBM Cloud®, Google Cloud®, Oracle Cloud® etc.

Services provided by a computing resources provider 728 include, but are not limited to, data analytics, data storage, archival storage, big data storage, virtual computing (including various scalable VM architectures), blockchain services, containers (e.g., application encapsulation), database services, development environments (including sandbox development environments), e-commerce solutions, game services, media and content management services, security services, server-less hosting, virtual reality (VR) systems, and augmented reality (AR) systems. Various techniques to facilitate such services include, but are not be limited to, virtual machines, virtual storage, database services, system schedulers (e.g., hypervisors), resource management systems, various types of short-term, mid-term, long-term, and archival storage devices, etc.

As may be contemplated, the systems such as service 730 and service 732 may implement versions of various services (e.g., the service 708 or the service 726) on behalf of, or under the control of, computing device 702 and/or computing device 724. Such implemented versions of various services may involve one or more virtualization techniques so that, for example, it may appear to a user of computing device 702 that the service 708 is executing on the computing device 702 when the service is executing on, for example, service 730. As may also be contemplated, the various services operating within the computing resources provider 728 environment may be distributed among various systems within the environment as well as partially distributed onto computing device 724 and/or computing device 702.

Client devices, user devices, computer resources provider devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things such as those described herein. The input devices can include, for example, a keyboard, a mouse, a keypad, a touch interface, a microphone, a camera, and/or other types of input devices including, but not limited to, those described herein. The output devices can include, for example, a display screen, a speaker, a haptic feedback system, a printer, and/or other types of output devices including, but not limited to, those described herein. A data storage device, such as a hard drive or flash memory, can enable the computing device to temporarily or permanently store data. A network interface, such as a wireless or wired interface, can enable the computing device to communicate with a network. Examples of computing devices (e.g., the computing device 702) include, but is not limited to, desktop computers, laptop computers, server computers, hand-held computers, tablets, smart phones, personal digital assistants, digital home assistants, wearable devices, smart devices, and combinations of these and/or other such computing devices as well as machines and apparatuses in which a computing device has been incorporated and/or virtually implemented.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

What is claimed is:

1. A computer-implemented method comprising:
receiving values for characteristics of an individual, wherein the characteristics include physical properties or medical history properties of the individual;
selecting a profile for the individual, wherein selecting includes using the values of the characteristics, wherein the profile specifies expected ranges for event data;
configuring a health monitoring application with the profile to generate notifications following an event exceeding an expected range;
collecting an event data set and a notification data set from the individual;
customizing the profile for the individual to form a custom profile, wherein customizing includes using a trained machine learning model to process a data set to output accurate notifications, wherein the data set includes the event data set, the notification data set, and a set of labels indicating the accuracy of notifications in the notification data set; and
configuring the health monitoring application with the custom profile.

2. The computer-implemented method of claim 1, further comprising:
detecting an anomalous event using the health monitoring application configured with the custom profile; and
sending a notification of the anomalous event.

3. The computer-implemented method of claim 2, wherein:
the event data set is a first event data set; and
detecting the anomalous event includes:
collecting a second event data set,
analyzing a first portion of the second event data set to determine a first baseline,
analyzing a second portion of the second event data set to determine a second baseline,
comparing the first baseline to the second baseline, and
determining that no baseline shift has occurred based on the comparison.

4. The computer-implemented method of claim 2, further comprising:
classifying the anomalous event by generating a severity score for the anomalous event.

5. The computer-implemented method of claim 2, wherein sending the notification of the anomalous event includes at least one of the following:
(a) sending a communication to the individual,
(b) sending a communication to a service monitoring the individual, or
(c) sending a communication to a contact designated by the individual.

6. The computer-implemented method of claim 5, further comprising:
detecting whether an acknowledgment of the communication is received,
calling a medical professional upon not detecting the acknowledgment within a time window.

7. The computer-implemented method of claim 1, wherein customizing the profile is preceded by:
calculating an accuracy score for notifications generated by the profile from the event data set, and
determining the accuracy score is less than a threshold value.

8. The computer-implemented method of claim 1, wherein configuring the health monitoring application with the custom profile includes:
calculating an accuracy score for notifications generated by the custom profile using the event data set, and
determining the accuracy score is greater than a threshold value.

9. The computer-implemented method of claim 1, further comprising:
detecting an anomalous event using the health monitoring application configured with the custom profile;
collecting a second event data set;
analyzing a first portion the second event data set to determine a first baseline;
analyzing a second portion of the second event data set to determine a second baseline;
comparing the first baseline to the second baseline; and
determining that a baseline shift has occurred based on the comparison.

10. The computer-implemented method of claim 9, further comprising:
prompting the individual to confirm the baseline shift.

11. The computer-implemented method of claim 9, further comprising:
sending a notification to the individual that the anomalous event is a result of the baseline shift.

12. The computer-implemented method of claim 1, wherein the event data includes heartrate data, step data, sleep data, activity data, appliance usage data, dietary data, medication data, location data, accelerometer data, blood pressure data, body temperature data, ambient temperature data, blood sugar data, or other sensor data.

13. The computer-implemented method of claim 1, wherein:
the profile is a first profile, and
selecting the first profile for the individual includes:
determining similarities between the individual and a plurality of profiles using the values of the characteristics, and
identifying the first profile as having the highest similarity with the individual among the plurality of profiles.

14. The computer-implemented method of claim 1, wherein:
the profile is a first profile, and
selecting the first profile for the individual includes generating the first profile from a plurality of profiles using the values of the characteristics.

15. A computer-readable medium on which computer-executable instructions are stored to implement a method comprising:
receiving values for characteristics of an individual, wherein the characteristics include physical properties or medical history properties of the individual;
selecting a profile for the individual, wherein selecting includes using the values of the characteristics, wherein the profile specifies expected ranges for event data;
configuring a health monitoring application with the profile to generate notifications following an event exceeding an expected range;
collecting an event data set and a notification data set from the individual;
customizing the profile for the individual to form a custom profile, wherein customizing includes using a trained machine learning model to process a data set to output accurate notifications, wherein the data set includes the event data set, the notification data set, and a set of labels indicating the accuracy of notifications in the notification data set; and
configuring the health monitoring application with the custom profile.

16. The computer-readable medium on of claim 15, wherein the method further comprises:
detecting an anomalous event using the health monitoring application configured with the custom profile; and
sending a notification of the anomalous event.

17. The computer-readable medium on of claim 16, wherein:
the event data set is a first event data set; and
detecting the anomalous event includes:
collecting a second event data set,
analyzing a first portion of the second event data set to determine a first baseline,
analyzing a second portion of the second event data set to determine a second baseline,
comparing the first baseline to the second baseline, and
determining that no baseline shift has occurred based on the comparison.

18. The computer-readable medium on of claim 16, wherein the method further comprises:
classifying the anomalous event by generating a severity score for the anomalous event.

19. The computer-readable medium on of claim 15, wherein customizing the profile is preceded by:
calculating an accuracy score for notifications generated by the profile from the event data set, and
determining the accuracy score is less than a threshold value.

20. A computing system including a processor and a memory storing instructions configured such that, when executed in cooperation with controlling the processor, the instructions operate the computing system to perform a method comprising:
receiving values for characteristics of an individual, wherein the characteristics include physical properties or medical history properties of the individual;
selecting a profile for the individual, wherein selecting includes using the values of the characteristics, wherein the profile specifies expected ranges for event data;
configuring a health monitoring application with the profile to generate notifications following an event exceeding an expected range;
collecting an event data set and a notification data set from the individual;
customizing the profile for the individual to form a custom profile, wherein customizing includes using a trained machine learning model to process a data set to output accurate notifications, wherein the data set includes the event data set, the notification data set, and a set of labels indicating the accuracy of notifications in the notification data set; and
configuring the health monitoring application with the custom profile.
